(19) United States
(12) Patent Application Publication
(10) Pub. No.: US 2013/0274339 A1
(43) Pub. Date: Oct. 17, 2013

(54) NOVEL FUNCTIONAL FOOD PRODUCT AND METHOD THEREOF

(71) Applicants: Leonid LURYA, Rehovot (IL); Vadim NOVODVORETS, Minsk (BY)

(72) Inventors: Leonid LURYA, Rehovot (IL); Vadim NOVODVORETS, Minsk (BY)

(21) Appl. No.: 13/864,441

(22) Filed: Apr. 17, 2013

Related U.S. Application Data
(60) Provisional application No. 61/625,093, filed on Apr. 17, 2012.

(51) Int. Cl. A23L 1/00; A61K 9/00

(52) U.S. Cl. CPC A23L 1/0073; A61K 9/0056
USPC 514/568; 426/650; 426/311; 426/536; 426/534; 426/532; 34/92; 514/772; 514/557

(57) ABSTRACT

A whole impregnated freeze dried food product for delivering a material of interest comprising;
a. moisture content of less than about 5%;
b. a material of interest; and,
c. taste additives;
   wherein, the food product is maintained in it's whole original structure, volume and shape; further wherein the food product is functionally impregnated with the material of interest in an amount equivalent to the removed moisture residual, and methods for obtaining same.

### FIELD OF THE INVENTION

This invention provides products and methods of functionally infusing or functionally impregnating materials of interest such as vitamins, herbal extracts, and medicinal into natural food products in their whole gross structural form. The resulting infused food products are consumable products for alleviating dietary insufficiency and, or to prevent or treat diseases or improve taste.

### BACKGROUND

The following prior art is believed to be the current status of the art:

EP patent document No. 144162 discloses methods for preparing intermediate moisture vegetables. The method includes the step of (1) pre-drying the vegetables to a moisture content of less than about 5 percent, (2) applying a low viscosity infusion cocktail comprising water, a polyhydric alcohol, and salt to the pre-dried vegetables, (3) equilibrating the pre-dried vegetables and infusion cocktail for at least about 30 minutes to provide the intermediate moisture vegetables, and (4) and packaging the intermediate moisture vegetables. These intermediate moisture vegetables provide appearance, texture, and taste similar to that provided by high quality cooked individually quick frozen vegetable.

U.S. Pat. No. 5,256,438 discloses preserved edible fruit products which consists of natural fruit or fruit pieces which have been dehydrated to a soluble solids level of 40% to 60%, a moisture level of 35% to 55% and a water activity level of 0.75 to 0.90 and to processes of producing such products and foods containing such products. The product remains unfrozen at temperatures within the range of 0 DEG C. to −25 DEG C.

WO2006017363 patent document discloses a method for preparing an expanded, vacuum puffed, dried fruit product. The method includes infusing the fruit with a low brix infusion solution and then expanding the fruit by subjecting it to a vacuum at elevated temperature followed by drying of the fruit under a vacuum at elevated temperature and finally cooling of the fruit under lowered temperature while maintaining the vacuum. The product produced by the method is light, crisp, and has a low water activity and a low buoyant density.

U.S. Pat. No. 6,440,449 discloses methods of infusing compositions. The process herein disclosed occur in aqueous environment, furthermore the infusion process is conducted by increasing the brix of the osmotic dehydration solution. The brix increasing process is carried out by adding osmotic dehydration solute to the osmotic dehydration solution, replacing at least part of the osmotic dehydration solution, or a combination thereof.

Other prior art provide modifications of food products with useful ingredients are available. Some of those prior art provide procedures of mixing vitamin extracts into a fresh fruit mass, e.g., Elliot Ritchie Alexander, “Vitamin Fruit Composition,” U.S. Pat. No. 1,886,931, and Clarence Birdseye, “Process of Improving and Preserving Food Products,” U.S. Pat. No. 2,419,877. Others involved adding flavors into frozen foodstuffs, such as those disclosed in Harry A. Noyes, “Process of Conditioning Foods,” U.S. Pat. No. 2,317,397. More recent work described using osmosis to dehydrate and preserve foods, e.g. Brimelow and Brittain, “Contiguous Food Impregnation,” U.S. Pat. No. 4,183,963, and Peter Butland. There have also been efforts made to infuse certain flavors into foods using osmosis, such as those disclosed in Lewis and Lewis, “Dehydrated Vegetables,” U.S. Pat. No. 4,683,141, Karim Nafisi-Novaghar, “Method of Making Dried Fruits,” U.S. Pat. No. 5,000,972, and Aebi, et al., “Process for Preparing Dehydrated Vegetable Products,” U.S. Pat. No. 5,368,873, and using a degradation enzyme, e.g. as in Poulose and Boston, “Enzyme Assisted Degradation of Surface Membranes of Harvested Fruits and Vegetables,” U.S. Pat. No. 5,037,662. Some have also explored methods of preserving fruits and vegetables using deep freezing, e.g., Berey et al., “Fruit and Vegetable Preservation Using Deep Freeze-by Complete or Partial Withdrawal of Free Water and Partial Replacement of it with Additives, Improving Appearance, Flavour and Shelf-life,” e.g., in Hungarian patent HU 47823.

Most of those prior art disclose procedures that occur and involve osmotic pressure and aqueous environment. None of this prior arts are satisfactory in terms of their ability to infuse maximum material of interest into food products.

There is therefore, a long felt unmet need to provide improved preparation methods of fast, tasty and healthy food product and more particularly, methods with efficient mechanisms for infusing food product with maximum content of material of interest while preserving the appearance and structure of the original food product.

### SUMMARY

There is provided in accordance with a preferred embodiment of the present invention a method for producing a freeze dried food product functionally impregnated with a material of interest, the method comprising steps of: (a) obtaining a fresh food product, (b) freeze drying the fresh food product to remove moisture content, (c) incubating dried food product in a solution containing material of interest, and (d) incubating of the wet food product with a soaked solution containing taste additives under normal pressure, wherein after step (b), the food product is substantially reduced in moisture to between about 0% and about 5%; the food product remains in it's original structure and shape; further wherein the food product is functionally impregnated with the material of interest after step d.

There is provided in accordance with a preferred embodiment of the present invention method for producing a freeze dried food product effective saturated with a material of interest, the method comprising steps of: (a) obtaining a fresh food product, (b) freeze drying the fresh food product to remove X moisture content from the food product Y, (c) incubating the dried food product in a solution containing material of interest, and (d) incubating of the wet food product with a soaked solution containing taste additives under normal pressure, wherein the food product is functionally impregnated with the material of interest in a hydromechanical manner such that (X/Y*100) equal Z % moisture content is reduced and Z % of the material of interest is infused; the food product remains in it's approximate original shape.

There is provided in accordance with a preferred embodiment of the present invention method for producing a freeze dried food product functionally impregnated with a material of interest, the method comprising steps of: (a) obtaining a fresh food product, (b) freeze drying the fresh food product to remove moisture content to between about 0% and about 5% of food product moisture volume, (c) incubating dried food product in a solution containing material of interest, and (d) incubating of the wet food product with a soaked solution containing taste additives under normal pressure, wherein the food product moisture is removed in a hydro mechanical manner by reducing the interior pressure of the food product and approximately equalizing the reduced pressure and the moisture deficit with an amount of the material of interest; the reduced moisture is determined by the differential in pressure from the external pressure exerted on the food product relative to the inner pressure of the food product.

There is further provided in accordance with a preferred embodiment of the present invention the method as defined above, wherein the step of drying the obtained food product is performed at a temperature of 40° C.-60° C. under normal pressure or under vacuum.

There is further provided in accordance with a preferred embodiment of the present invention the method as defined above, wherein the step of freeze drying the fresh food product is performed by combining vacuum process to the food product to remove moisture content.

There is further provided in accordance with a preferred embodiment of the present invention the method as defined above, wherein the incubation in step C is performed under vacuum.

There is further provided in accordance with a preferred embodiment of the present invention the method as defined above, further comprising the step of drying the soaked food product;

There is further provided in accordance with a preferred embodiment of the present invention the method as defined above, wherein the food product is dehydrated in step (b) until the residual moisture extent is between 0 to 5%.

There is further provided in accordance with a preferred embodiment of the present invention the method as defined above, wherein the residual moisture extent is selected from a group consisting of: 0%, 1%, 2%, 3%, 4%, 5% of the food product volume.

There is further provided in accordance with a preferred embodiment of the present invention the method as defined above, wherein the material of interest is a natural pharmacological or bio-active material containing a definite taste.

There is further provided in accordance with a preferred embodiment of the present invention the method as defined above, wherein the material of interest includes phytochemicals and, or nutraceutical material

There is further provided in accordance with a preferred embodiment of the present invention the method as defined above, wherein the nutraceutical material is selected from a group consisting of: vitamins, minerals, isoflavoronals, lycopene, resveratol, indocarbonals, anthocyanins, soluble fiber, high protein rice, and soy isolate or any combination thereof.

There is further provided in accordance with a preferred embodiment of the present invention the method as defined above, wherein the food product further comprises food additives selected from a group consisting of acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners.

There is further provided in accordance with a preferred embodiment of the present invention the method as defined above, wherein the taste additives are volatile compounds selected from the group consisting of esters, aromatic compounds, linear terpenes, cyclic terpenes, amines, aldehydes, ketones, lactones, thiols, miscellaneous compounds alcohols or any combination thereof.

There is further provided in accordance with a preferred embodiment of the present invention the method as defined above, wherein the material of interest may be any none material for alleviating dietary insufficiency and, or to prevent or treat diseases such as cancer, heart disease, Alzheimer's disease.

There is further provided in accordance with a preferred embodiment of the present invention the method as defined above, wherein the solution is an aqueous infusion solution, further comprises edible acid selected from the group consisting of citric acid, malic acid, fumaric acid, ascorbic acid, erythorbic acid, phosphoric acid, and salts thereof.

There is further provided in accordance with a preferred embodiment of the present invention the method as defined above, wherein the solution further comprises an edible anti-microbiological agent selected from the group consisting of benzoic acid, sorbic acid, propionic acid and salts thereof.

There is further provided in accordance with a preferred embodiment of the present invention the method as defined above, wherein the solution further comprises preservatives.

There is further provided in accordance with a preferred embodiment of the present invention the method as defined above, wherein the food product is selected from a group consisting of: vegetables, fruits, fishes.

There is further provided in accordance with a preferred embodiment of the present invention the method as defined above, wherein the composition is a medicinal capable of providing a medical or dietary benefit to a human.

There is further provided in accordance with a preferred embodiment of the present invention the method as defined above, wherein the composition is a medicinal capable of providing a medical or dietary benefit to a non-human animal.

There is further provided in accordance with a preferred embodiment of the present invention the method as defined above, wherein the infusion solution further comprises about polyhydric alcohol.

There is further provided in accordance with a preferred embodiment of the present invention the method as defined above, wherein the polyhydric alcohol is selected from glycerol, propylene glycol, and mixtures thereof.

There is further provided in accordance with a preferred embodiment of the present invention the method as defined above, wherein the vacuum and temperature used during the freeze drying step preserves the product whole shape, structure and composition.

There is further provided in accordance with a preferred embodiment of the present invention the method as defined above, wherein the impregnating solution is a solution which comprises at least one of sugar, salt, a meat curing agent and a humectant.

There is further provided in accordance with a preferred embodiment of the present invention the method as defined above, wherein the humectant is one or more substances selected from the group consisting of dextrose, glycerol, sorbitol, salt, fructose, sucrose and mannitol.

There is further provided in accordance with a preferred embodiment of the present invention the method as defined above, wherein the humectant solution comprises a solution of two or more substances selected from the group consisting of dextrose, glycerol, sorbitol, salt, fructose, sucrose, propylene glycol and mannitol.

There is provided in accordance with a preferred embodiment of the present invention a whole impregnated freeze dried food product for delivering a material of interest comprising; (a) moisture content of less than about 5%, (b) a material of interest; and (c) taste additives, wherein, the food product is maintained in it's whole original structure, volume and shape; further wherein the food product is functionally impregnated with the material of interest such as in an amount equivalent to the removed moisture residual.

There is further provided in accordance with a preferred embodiment of the present invention the food product as defined above, wherein the freeze dried food product is obtained at a temperature of 40° C.-60° C. under ambient pressure or under vacuum.

There is further provided in accordance with a preferred embodiment of the present invention the food product as defined above, wherein the food product is dehydrated until the residual moisture extent is of 0 to 5%.

There is further provided in accordance with a preferred embodiment of the present invention the food product as defined above, wherein the material of interest is a natural pharmacological or bio-active material containing a definite taste.

There is further provided in accordance with a preferred embodiment of the present invention the food product as defined above, wherein the material of interest may be any none material for alleviating dietary insufficiency and, or to prevent or treat diseases such as cancer, heart disease, Alzheimer's disease.

There is further provided in accordance with a preferred embodiment of the present invention the food product as defined above, wherein the material of interested are infused by incubation of the dried food product in an impregnating solution under vacuum.

There is further provided in accordance with a preferred embodiment of the present invention the food product as defined above, wherein the impregnating solution is an aqueous infusion solution, further comprises edible acid selected from the group consisting of citric acid, malic acid, fumaric acid, ascorbic acid, erythorbic acid, phosphoric acid, and salts thereof.

There is further provided in accordance with a preferred embodiment of the present invention the food product as defined above, wherein the solution further comprises an edible anti-microbiological agent selected from the group consisting of benzoic acid, sorbic acid, propionic acid and salts thereof.

There is further provided in accordance with a preferred embodiment of the present invention the food product as defined above, wherein the solution further comprises preservatives.

There is further provided in accordance with a preferred embodiment of the present invention the food product as defined above, wherein the taste additives are infused by incubation of the wet food product with a soak solution under normal pressure.

There is further provided in accordance with a preferred embodiment of the present invention the food product as defined above, wherein the food product is selected from a group consisting of: vegetables, fruits, fishes and meat.

There is further provided in accordance with a preferred embodiment of the present invention the food product as defined above, wherein the natural or bio-active material is a nutraceutical material selected from a group consisting of: vitamins, minerals, isoflavoronals, lycopene, resveratol, indocarbonals, anthocyanins, soluble fiber, high protein rice, and soy isolate or any combination thereof.

There is further provided in accordance with a preferred embodiment of the present invention the food product as defined above, wherein the food product further comprises food additives selected from a group consisting of acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners.

There is further provided in accordance with a preferred embodiment of the present invention the food product as defined above, wherein the taste additives are volatile compounds selected from the group consisting of esters, aromatic compounds, linear terpenes, cyclic terpenes, amines, aldehydes, ketones, lactones, thiols, miscellaneous compounds alcohols or any combination thereof.

There is further provided in accordance with a preferred embodiment of the present invention the food product as defined above, wherein the material of interest is a medicinal capable of providing a medical or dietary benefit to a human.

There is further provided in accordance with a preferred embodiment of the present invention the food product as defined above, wherein the material of interest is a medicinal capable of providing a medical or dietary benefit to a non-human animal.

There is further provided in accordance with a preferred embodiment of the present invention the food product as defined above, wherein the impregnating solution further comprises about polyhydric alcohol.

There is further provided in accordance with a preferred embodiment of the present invention the food product as defined above, wherein the polyhydric alcohol is selected from glycerol, propylene glycol, and mixtures thereof.

There is further provided in accordance with a preferred embodiment of the present invention the food product as defined above, wherein the impregnating solution is a solution which comprises at least one of sugar, salt, a meat curing agent and a humectant.

There is further provided in accordance with a preferred embodiment of the present invention the food product as defined above, wherein the humectant is one or more substances selected from the group consisting of dextrose, glycerol, sorbitol, salt, fructose, sucrose and mannitol.

There is further provided in accordance with a preferred embodiment of the present invention the food product as defined above, wherein the humectant solution comprises a solution of two or more substances selected from the group consisting of dextrose, glycerol, sorbitol, salt, fructose, sucrose, propylene glycol and mannitol. There is further provided in accordance with a preferred embodiment of the present invention, a production line for producing a freeze dried food product functionally impregnated with a material of interest, said production line comprising: (a) an instruction protocol for operating said production line (b) a lyophilizer adapted for freeze-drying a food product, (c) an incubator adapted for incubating said food product in solution containing material of interest under vacuum and further adapted for incubating with solution containing taste additives under normal pressure, (d) a drier adapted for drying the food product after an incubation step, wherein said production line provides said food product substantially reduced in moisture to between about 0% and about 5%. The food product remaining in it's original structure and shape; further wherein the food product is functionally impregnated with the material of interest after applying the incubator.

There is further provided in accordance with a preferred embodiment of the present invention, wherein the instruction protocol is encoded in a computer readable medium or written on printed matter or represented on a computer screen.

There is further provided in accordance with a preferred embodiment of the present invention, a computer readable medium comprising instructions for causing a computer to operate a production line producing a freeze dried food product functionally impregnated with a material of interest, by the method comprising steps of: (a) freeze drying a fresh food product to remove moisture content;

(b) incubating dried food product in a solution containing material of interest, and;

(c) incubating of the wet food product with a soaked solution containing taste additives under normal pressure, wherein after step (b), the food product is substantially reduced in moisture to between about 0% and about 5%; the food product remains in it's original structure and shape; further wherein the food product is functionally impregnated with the material of interest after step d.

### BRIEF DESCRIPTION

In order to understand the invention and to see how it may be implemented in practice, a few preferred embodiments will now be described, by way of non-limiting example only, with reference to be accompanying drawings, in which:

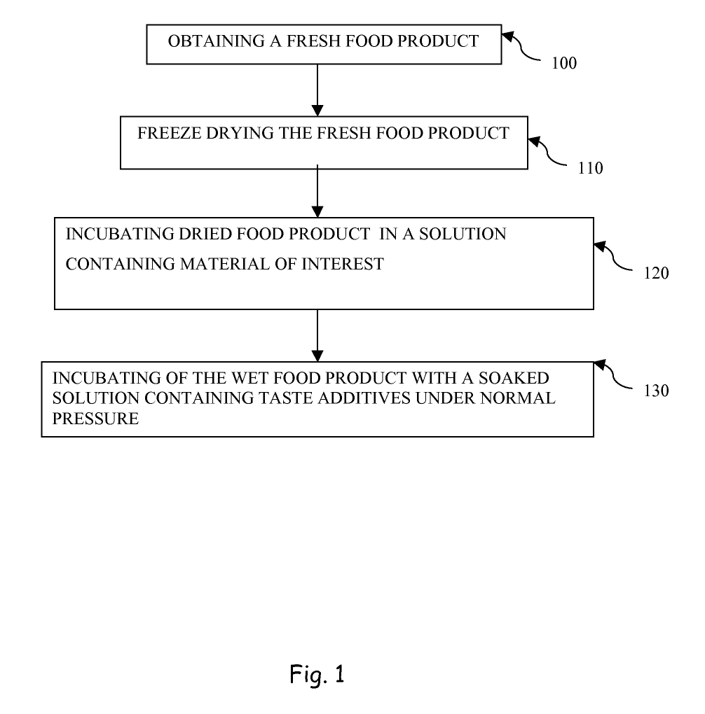

FIG. 1 is a schematic diagram of the process of impregnating material of interest to food product, in accordance with a preferred embodiment of the present invention.

### DETAILED DESCRIPTION

The following description is provided so as to enable any person skilled in the art to make use of the invention and sets forth the best modes contemplated by the inventor of carrying out this invention. Various modifications, however, will remain apparent to those skilled in the art, since the generic principles of the present invention have been defined specifically to provide product and method of impregnating material of interest to food product while preserving its original shape and structure.

The term “food product(s),” as used herein, is intended to cover all edible material of origin including, vegetable, fruit, fish, meat or any natural edible material known in the art.

The present invention relates to processes for producing a freeze dried food product which are functionally impregnated with a material of interest such as vitamins, herbal extracts, nutrients, medicinal and any other composition of interest which is capable of being infused for supplementing the characteristics and properties of the food and can be consumed by humans or animals. The method disclosed herein can be used to provide dietary benefits and, or to prevent and, or to treat diseases. The procedure provide effective saturation of vegetables with any natural or bio-active material.

The present invention provides a method for treating fresh vegetables so that the important qualities such as natural appearance, flavor, taste, and product integrity will be retained while the shelf-life of the product is appreciably extended as compared to the untreated product.

The method for producing a freeze dried food product functionally impregnated with a material of interest, comprising steps of: (a) obtaining a fresh food product 100, (b) freeze drying the fresh food product 110, by combining a vacuum process to the food product to remove moisture content, (c) incubating dried food product in a solution containing material of interest 120, and (d) incubating of the wet food product with a soaked solution containing taste additives under normal pressure 130.

After step (c), the food product is substantially reduced in moisture to between about 0% and about 5%. The food product is functionally impregnated with a material of interest. The product contains the material of interest such as in an amount equivalent to the removed moisture residual while the food product remains in it's original structure and shape. The preferred stage of moisture removal will vary from product to product regarding time, temperature and pressure.

In accordance with the present invention a method for producing a freeze dried food product effective saturated with a material of interest is presented, the method comprising steps of: (a) obtaining a fresh food product, (b) freeze drying the fresh food product to remove X moisture content from the food product Y, (c) incubating the dried food product in a solution containing material of interest, and

(d) incubating of the wet food product with a soaked solution containing taste additives under normal pressure, wherein the food product is functionally impregnated with the material of interest in a hydromechanical manner such that (X/Y*100) equal Z % moisture content is reduced and Z % of the material of interest is infused. The food product remains in it's approximate original shape.

In accordance with the present invention, a method for producing a freeze dried food product functionally impregnated with a material of interest is presented, the method comprising the steps of: (a) obtaining a fresh food product, (b) freeze drying the fresh food product to remove moisture content to between about 0% and about 5% of food product moisture volume, (c) incubating dried food product in a solution containing material of interest, and (d) incubating of the wet food product with a soaked solution containing taste additives under normal pressure, wherein the food product moisture is removed in a hydro mechanical manner by reducing the interior pressure of the food product and approximately equalizing the reduced pressure and the moisture deficit with an amount of the material of interest. The reduced moisture is determined by the differential in pressure from the external pressure exerted on the food product relative to the inner pressure of the food product.

The method further comprises the step of drying the wet food product depending on the food product type.

Freeze-drying also known as lyophilization, lyophilization or cryodesiccation is used as a dehydration process to preserve a perishable material. Freeze-drying works by freezing the material and then reducing the surrounding pressure to allow the frozen water in the material to sublimate directly from the solid phase to the gas phase.

In a smaller scale, the freezing is done by placing the material in a freeze-drying flask and rotating the flask in a bath, called a shell freezer, which is cooled by mechanical refrigeration, dry ice and methanol, or liquid nitrogen. On a larger scale, freezing is usually done using a freeze-drying machine. In some embodiments of the present invention, the process will include steps of batch lyophilisation or continuous lyophilisation. Industrial rotary lyophilisers, manifold lyophilisers or traystyle lyophilisers or any other conventional lyophilisation equipment may be used adapted for the purpose may be used.

In the drying phase, the pressure is lowered to the range of a few millibars, and enough heat is supplied to the food product for sublime the water. The amount of heat necessary is calculated using the sublimating molecules' latent heat of sublimation. In this initial drying phase, about 95% of the water in the material is sublimated (This phase is carried for several days).

All food products contain moisture. The process of dehydration and infusion are limited by the differential in pressure from outside the food product and a lesser pressure from within the food product. The present invention is based on hydro mechanical process. The food product moisture is removed in a hydro mechanical manner by overcoming the food product's inner pressure and approximately equalizing the reduced moisture delta with an amount of material of interest. The step of removing moisture is limited by the differential in pressure from outside the food product and a lesser pressure from within the food product.

The infusion process includes freezing the food, then removing almost all the moisture in a vacuum chamber at an ambient pressure. The process starts by freeze drying the food product to almost 0% residual moisture. The freeze-dried product can be thought of as a dry sponge with voids where moisture previously was and which has little resistance from absorbing media. During all of the process the food product maintains its original structure.

The freezing process may be performed by using a blast freezer (also known as a shock freezer), or even by using liquid nitrogen or any known instrument or process which rapidly brings the temperature of foods or vegetables down and freezes them extremely quickly without creating any ice crystals or creating very small ice crystals.

The drying conditions must be sufficient to reduce the water content in the finished product sufficiently to produce the amorphous film necessary to protect the vegetable product. It has been found that at a water content of about 6% or less such amorphous films are formed. The process involves drying below about 2% which does not damage the flavor and does not cause texture degradation. Conventional drying techniques such as air drying, convection drying, vacuum drying, freeze drying, solar drying and the like may be employed to achieve the desired final water content.

The infusion of the vegetables may take place at ambient pressure or it may be carried out under vacuum. Vacuum infusion methods of the type which may be used in the invention are well-known to those skilled in the art. Preferably, vacuum infusion is carried out by subjecting the vegetable matter in the infusion solution to a vacuum of from 1.33 to 5.33 kPa (10 to 40 mmHg) for a period of from about 1 to 10 minutes. A typical vacuum infusion process involves holding the vegetable matter in the infusion solution, applying a vacuum of about 20 mmHg for about 5 minutes, slowly releasing the vacuum and holding for a further 5 minutes.

Optionally, the infusion is speeded up by the use of heat, i.e., from ranges of 20-80 deg. C., optionally 30-70 deg. C., and generally 40-60 deg. C., and preferably at about 50 [deg.] C., and a high strength of solution, e.g., 77 deg. B., to under 24 hours. Stirring of the solution is optionally used to speed the infusion.

The method of the present invention can be performed at various temperatures, such as at about room temperature or up to about 50 deg. C. to thin out the dehydration solution for greater absorption.

In accordance with the present invention, incubation of the dried food products in solution containing dissolved material of interest under vacuum, is performed. The material of interest is a natural pharmacological or bio-active material containing a definite taste. The material of interest may be any known material for alleviating dietary insufficiency and, or to prevent or treat diseases such as cancer, heart disease, Alzheimer's disease. The material of interest may be a medicinal composition which is capable of providing a medical or dietary benefit to a human or, and to an animal. The solution includes material of interest such as phytochemicals and,or nutraceutical material. The nutraceutical material is selected from a group consisting of: vitamins, minerals, isoflavoronals, lycopene, resveratol, indocarbonals, anthocyanins, soluble fiber, high protein rice, and soy isolate or any combination thereof. The solution is an aqueous infusion solution, further comprises edible acid selected from the group consisting of citric acid, malic acid, fumaric acid, ascorbic acid, erythorbic acid, phosphoric acid, and salts thereof. The solution may further comprises preservatives known in the art and, or edible anti-microbiological agents selected from the group consisting of benzoic acid, sorbic acid, propionic acid and salts thereof.

In accordance with the present invention, the infusion or impregnating solution further comprises about polyhydric alcohol. The polyhydric alcohol is selected from glycerol, propylene glycol, and mixtures thereof. The impregnating solution is a solution which comprises at least one of sugar, salt, a meat curing agent and a humectant.

The humectant is one or more substances selected from the group consisting of dextrose, glycerol, sorbitol, salt, fructose, sucrose and mannitol.

The humectant solution comprises a solution of two or more substances selected from the group consisting of dextrose, glycerol, sorbitol, salt, fructose, sucrose, propylene glycol and mannitol.

In accordance with the present invention, the temperature conditions under which infusion with a solution occurs has been found to range from the freezing point of the infusion solution up to about the solution's boiling point, preferably from about 45 DEG F. to about 220 DEG F., however optimal temperature ranges vary by product. Higher soaking temperatures from about 140 DEG F. to about 220 DEG F. are suitable for certain green leafy vegetables such as spinach for stabilization of color, although acceptable product has been produced with soaking at temperatures down to about 45 DEG F. Lower soaking temperatures are generally employed for vegetables that have enzymatically generated flavor such as onion and garlic. A preferred range for these products is from about 45 DEG F. to about 160 DEG F.

The infusion time need to be long enough to ensure coating and infusion. Excessive infusion times which cause degradation of the vegetable should be avoided. Times on the order of a few seconds up to several hours are suitable, preferably from 20-30 seconds to about two hours.

In accordance with the present invention, incubation of the wet food product with the soaked solution containing taste additives under normal pressure, is also performed. The taste additives may include flavor, or color or any combination thereof. The taste additives are volatile compounds selected from the group consisting of esters, aromatic compounds, linear terpenes, cyclic terpenes, amines, aldehydes, ketones, lactones, thiols, miscellaneous compounds alcohols or any combination thereof.

The soaked solution includes a taste additives such as sugar component such as high fructose corn syrup. Typically, it includes at least about 35% to about 100% fructose, and preferably about 42% to about 90% fructose. The balance of the sugar may be comprised of dextrose or any of a number of saccharide materials including monosaccharides, disaccharides and polysaccharides and their degradation products, e.g., pentoses including aldopentoses, ketopentoses like xylose and arabinose, a deoxyaldose like rhamnose, hexoses and reducing saccharides such as aldohexoses like glucose, galactose and mannose; the ketohexuloses, like sorbose and xylulose; disaccharides, like maltulose, lactose and maltose; nonreducing disaccharides such as a sucrose, other polysaccharides such as dextrin and raffinose, and hydrolyzed starches which contain as their constituents oligosaccharides. The balance of the sugar may be of a low molecular weight so as to offer a substantial effect in increasing the osmotic pressure of the sugar solution. The balance of the sugar may also be comprised of polyhydric alcohols such as glycerol and the like. The polyhydric alcohols preferably comprise only about 1 to about 10% of the sugar component.

In accordance with the present invention, the method further comprises the step of drying of the obtained food product at a temperature 40° C.-60° C. under normal pressure or under the vacuum. The final step of drying may be achieved in either a conventional continuous dryer or in a bin dryer. The dryer allows the more even final drying of pieces of food products of different sizes to a more uniform final moisture content. The final rapid drying of the vegetables is achieved to a predetermined moisture content (however uniform equilibration of water activity throughout the pieces occurs during storage). The drying process depends on the type of the food product.

Further wherein the food product is functionally impregnated with the material of interest.

The product resulting from the processing steps has a long shelf-life, in some cases over one year at ambient temperatures in sealed packages.

In accordance with the present invention, an impregnating freeze dried food product is provided for delivering a material of interest comprising; (a) a moisture in an amount between about 0% and about 5%, (b) a material of interest; and (c) taste additives. In the aforementioned, the food product contains the material of interest such as in an amount equivalent to the removed moisture residual to between about 0% and about 5% while the food product remains and maintains it's whole original structure, volume and shape. The food product is functionally impregnated with material of interest after it is substantially reduced in moisture to between about 0% and about 5%. The product is produced in a technology that maintains its original structure and shape, better its texture, color, and reduces its levels of oxidation.

The food product is immersed in a concentrated solution, such as brine, syrup, meat curing solutions humectant solutions and mixtures thereof.

The solution may contain one solute, for example sodium chloride or it may contain a mixture of two or more solutes, for instance, sodium chloride and dextrose. In addition, at food product is incubated with taste additives soaked solution, food additives such as flavorings, coloring substances, seasoning, preservatives, nutritional supplements and anti-oxidants, etc. may optionally be added. The food additives may further comprises acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners

The freeze dried food product is obtained at a temperature of 40° C.-60° C. under ambient pressure or under vacuum.

The food product is dehydrated until the residual moisture extent is of 0 to 5%.

The residual moisture extent is selected from a group consisting of: 0%, 1%, 2%, 3%, 4%, 5% of the food product volume.

The food product material of interest is a natural pharmacological or bio-active material containing a definite taste. The material of interest may be any none material for alleviating dietary insufficiency and, or to prevent or treat diseases such as cancer, heart disease, Alzheimer's disease. The material of interested are infused by incubation of the dried food product in an impregnating solution under vacuum.

The food product impregnating solution is an aqueous infusion solution, further comprises edible acid selected from the group consisting of citric acid, malic acid, fumaric acid, ascorbic acid, erythorbic acid, phosphoric acid, and salts thereof.

There is further provided in accordance with a preferred embodiment of the present invention, the food product solution further comprises an edible anti-microbiological agent selected from the group consisting of benzoic acid, sorbic acid, propionic acid and salts thereof. The food product further comprises preservatives.

The food product further comprises taste additives which are infused by incubation of the wet food product with a soak solution under normal pressure. The taste additives are volatile compounds selected from the group consisting of esters, aromatic compounds, linear terpenes, cyclic terpenes, amines, aldehydes, ketones, lactones, thiols, miscellaneous compounds alcohols or any combination thereof.

The food product further comprises food additives selected from a group consisting of acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners.

The food product natural or bio-active material is a nutraceutical material selected from a group consisting of: vitamins, minerals, isoflavoronals, lycopene, resveratol, indocarbonals, anthocyanins, soluble fiber, high protein rice, and soy isolate or any combination thereof.

There is further provided in accordance with a preferred embodiment of the present invention, the food product's material of interest is a medicinal capable of providing a medical or dietary benefit to a human. The material of interest is a medicinal capable of providing a medical or dietary benefit to a non-human animal.

There is further provided in accordance with a preferred embodiment of the present invention, the food product infusion solution further comprises about polyhydric alcohol. The polyhydric alcohol is selected from glycerol, propylene glycol, and mixtures thereof.

The food product solution is a solution which comprises at least one of sugar, salt, a meat curing agent and a humectant. The humectant is one or more substances selected from the group consisting of dextrose, glycerol, sorbitol, salt, fructose, sucrose and mannitol. The humectant solution comprises a solution of two or more substances selected from the group consisting of dextrose, glycerol, sorbitol, salt, fructose, sucrose, propylene glycol and mannitol.

There is further provided in accordance with a preferred embodiment of the present invention, the freeze dried food product as defined above is obtained at a temperature of 40° C.-60° C. under ambient pressure or under vacuum.

There is further provided in accordance with a preferred embodiment of the present invention, the food product as defined above is dehydrated until the residual moisture extent is of 0 to 5%. The residual moisture extent is selected from a group consisting of: 0%, 1%, 2%, 3%, 4%, 5% of the food product volume.

There is further provided in accordance with a preferred embodiment of the present invention, the material of interest as defined above is infused by incubation of the dried food product in an impregnating solution under vacuum.

There is further provided in accordance with a preferred embodiment of the present invention, the solution further comprises an edible anti-microbiological agent selected from the group consisting of benzoic acid, sorbic acid, propionic acid and salts thereof.

There is further provided in accordance with a preferred embodiment of the present invention, the solution further comprises preservatives. There is further provided in accordance with a preferred embodiment of the present invention, the taste additives are infused by incubation of the wet food product with a soak solution under normal pressure.

The method production process may be as a continuous production or a bath production. In Continuous production which is also known as continuous process or a continuous flow process, or a semi-continuous processes the materials, either dry bulk or fluids that are being processed are continuously in motion, undergoing chemical reactions or subject to mechanical or heat treatment. Continuous usually means operating 24 hours per day, seven days per week with infrequent maintenance shutdowns, such as semi-annual or annual.

In batch production the object in question is created stage by stage over a series of workstations.

There is further provided in accordance with a preferred embodiment of the present invention, the taste additives further comprises a flavor, or a color.

There is further provided in accordance with a preferred embodiment of the present invention, a production line for producing a freeze dried food product functionally impregnated with a material of interest, the production line comprising: (a) an instruction protocol for operating the production line (b) a lyophilizer adapted for freeze-drying a food product, (c) an incubator adapted for incubating the food product in solution containing material of interest under vacuum and further adapted for incubating with solution containing taste additives under normal pressure, (d) a drier adapted for drying the food product after an incubation step, wherein the production line provides the food product substantially reduced in moisture to between about 0% and about 5%; the food product remaining in it's original structure and shape; further wherein the food product is functionally impregnated with the material of interest after applying the incubator.

There is further provided in accordance with a preferred embodiment of the present invention, wherein the instruction protocol is encoded in a computer readable medium or written on printed matter or represented on a computer screen.

There is further provided in accordance with a preferred embodiment of the present invention, a computer readable medium comprising instructions for causing a computer to operate a production line producing a freeze dried food product functionally impregnated with a material of interest, by the method comprising steps of: (a) freeze drying a fresh food product to remove moisture content,

(b) incubating dried food product in a solution containing material of interest, and

(c) incubating of the wet food product with a soaked solution containing taste additives under normal pressure, wherein after step (b), the food product is substantially reduced in moisture to between about 0% and about 5%, the food product remains in it's original structure and shape. Further wherein the food product is functionally impregnated with the material of interest after step d.

In the foregoing description, embodiments of the invention, including preferred embodiments, have been presented for the purpose of illustration and description. They are not intended to be exhaustive or to limit the invention to the precise form disclosed. Obvious modifications or variations are possible in light of the above teachings. The embodiments were chosen and described to provide the best illustration of the principals of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A method for producing a freeze dried food product functionally impregnated with a material of interest, wherein said method is selected from the group consisting of
a. steps of;
i. obtaining a fresh food product;
ii. freeze drying said fresh food product to remove moisture content;
iii. incubating dried food product in a solution containing material of interest, and;
iv. incubating of the wet food product with a soaked solution containing taste additives under normal pressure
wherein after step ii, said food product is substantially reduced in moisture to between about 0% and about 5%; said food product remains in it's original structure and shape; further wherein said food product is functionally impregnated with said material of interest after step iv. or said method comprises
b. steps of:
i. obtaining a fresh food product;
ii. freeze drying said fresh food product to remove moisture content to between about 0% and about 5% of food product moisture volume;
iii. incubating dried food product in a solution containing material of interest, and;
iv. incubating of the wet food product with a soaked solution containing taste additives under normal pressure;
wherein said food product moisture is removed in a hydro mechanical manner by reducing the interior pressure of said food product and approximately equalizing said reduced pressure and said moisture deficit with an amount of said material of interest; said reduced moisture is determined by the differential in pressure from the external pressure exerted on said food product relative to the inner pressure of said food product or said method comprises
c. steps of
i. obtaining a fresh food product;
ii. freeze drying said fresh food product to remove X moisture content from said food product Y;
iii. incubating the dried food product in a solution containing material of interest, and;
iv. incubating of the wet food product with a soaked solution containing taste additives under normal pressure;
wherein said food product is functionally impregnated with said material of interest in a hydromechanical manner such that (X/Y*100) equal Z % moisture content is reduced and Z % of said material of interest is infused; said food product remains in it's approximate original shape.

2. The method according to claim 1, wherein said step of drying the obtained food product is performed at a temperature of about 40° C.-about 60° C. under normal pressure or under vacuum.

3. The method according to claim 1, wherein said step of freeze drying said fresh food product is performed by combining vacuum process to said food product to remove moisture content.

4. The method according to claim 1, wherein said incubation in step iii is performed under vacuum.

5. The method according to claim 1, further comprising the step of drying the soaked food product;

6. The method according to claim 1, wherein said food product is dehydrated in step (b) until the residual moisture extent is between about 0% to about 5%.

7. The method according to claim 1, wherein said residual moisture extent is selected from a group consisting of: about 0%, about 1%, about 2%, about 3%, about 4%, about 5% of said food product volume.

8. The method according to claim 1, wherein at least one of the following is true;
a. said material of interest is a natural pharmacological or bio-active material containing a definite taste
b. said material of interest includes any of phytochemicals or nutraceutical material, said nutraceutical material selected from a group consisting of; vitamins, minerals, isoflavoronals, lycopene, resveratol, indocarbonals, anthocyanins, soluble fiber, high protein rice, and soy isolate or any combination thereof
c. said food product further comprises food additives selected from a group consisting of acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners
d. said taste additives are volatile compounds selected from the group consisting of esters, aromatic compounds, linear terpenes, cyclic terpenes, amines, aldehydes, ketones, lactones, thiols, miscellaneous compounds alcohols or any combination thereof
e. said material of interest may be any known material for alleviating dietary insufficiency and, or to prevent or treat diseases such as cancer, heart disease, Alzheimer's disease, gastric disorder
f. said solution is an aqueous infusion solution, further comprising edible acid selected from the group consisting of citric acid, malic acid, fumaric acid, ascorbic acid, erythorbic acid, phosphoric acid, and salts thereof
g. said solution further comprises an edible anti-microbiological agent selected from the group consisting of benzoic acid, sorbic acid, propionic acid and salts thereof
h. said solution further comprises preservatives
i. said food product is selected from a group consisting of: vegetables, fruits, fishes
j. composition is a medicinal capable of providing a medical or dietary benefit to a human
k. Said composition is a medicinal capable of providing a medical or dietary benefit to a non-human animal
l. said the infusion solution further comprises polyhydric alcohol, said polyhydric alcohol selected from glycerol, propylene glycol, and mixtures thereof
m. said vacuum and temperature used during the freeze drying step preserves the product whole shape, structure and composition
n. said impregnating solution is a solution which comprises at least one of sugar, salt, a meat curing agent and a humectant and said humectant is one or more substances selected from the group consisting of dextrose, glycerol, sorbitol, salt, fructose, sucrose and mannitol
o. said solution comprises a solution of two or more substances selected from the group consisting of dextrose, glycerol, sorbitol, salt, fructose, sucrose, propylene glycol and mannitol

9. A whole impregnated freeze dried food product for delivering a material of interest comprising;
a. moisture content of less than about 5%;
b. a material of interest; and,
c. taste additives;
wherein, said food product is maintained in it's whole original structure, volume and shape; further wherein said food product is functionally impregnated with said material of interest such as in an amount equivalent to the removed moisture residual.

10. The food product according to claim 9, wherein at least one of the following is true;
a. said freeze dried food product is obtained at a temperature of about 40° C.-about 60° C. under ambient pressure or under vacuum.
b. said food product is dehydrated until the residual moisture extent is of about 0 to about 5%.
c. said material of interest is a natural pharmacological or bio-active material containing a definite taste
d. said material of interest may be any material for alleviating dietary insufficiency or to prevent or treat diseases such as cancer, heart disease, Alzheimer's disease
e. said material of interest is infused by incubation of said dried food product in an impregnating solution under vacuum
f. said impregnating solution is an aqueous infusion solution, further comprises edible acid selected from the group consisting of citric acid, malic acid, fumaric acid, ascorbic acid, erythorbic acid, phosphoric acid, and salts thereof

11. The food product according to claim 9, wherein at least one of the following is true;
a. said solution further comprises an edible anti-microbiological agent selected from the group consisting of benzoic acid, sorbic acid, propionic acid and salts thereof
b. said solution further comprises preservatives
c. said taste additives are infused by incubation of the wet food product with a soak solution under normal pressure

12. The food product according to claim 9, wherein at least one of the following is true;
a. said food product is selected from a group consisting of: vegetables, fruits, fishes and meat
b. said natural or bio-active material is a nutraceutical material selected from a group consisting of: vitamins, minerals, isoflavoronals, lycopene, resveratol, indocarbonals, anthocyanins, soluble fiber, high protein rice, and soy isolate or any combination thereof.
c. said food product further comprises food additives selected from a group consisting of acids, acidity regulators, anticaking agents, antifoaming agents, antioxidants, bulking agents, food coloring, color retention agents, emulsifiers, flavors, flavor enhancers, flour treatment agents, glazing agents, humectants, tracer gas, stabilizers, preservatives, sweeteners, thickeners.
d. said taste additives are volatile compounds selected from the group consisting of esters, aromatic compounds, linear terpenes, cyclic terpenes, amines, aldehydes, ketones, lactones, thiols, miscellaneous compounds alcohols or any combination thereof

13. The food product according to claim 9, wherein said material of interest is a medicinal capable of providing a medical or dietary benefit to a human.

14. The food product according to claim 9, wherein said material of interest is a medicinal capable of providing a medical or dietary benefit to a non-human animal.

15. The food product according to claim 10, wherein said impregnating solution further comprises polyhydric alcohol.

16. The food product according to claim 15, wherein said polyhydric alcohol is selected from glycerol, propylene glycol, and mixtures thereof

17. The food product according to claim 10, wherein said impregnating solution is a solution which comprises at least one of sugar, salt, a meat curing agent and a humectant.

18. The food product according to claim 17, wherein said humectant is one or more substances selected from the group consisting of dextrose, glycerol, sorbitol, salt, fructose, sucrose and mannitol.

19. The food product according to claim 17, wherein said humectant solution comprises a solution of two or more substances selected from the group consisting of dextrose, glycerol, sorbitol, salt, fructose, sucrose, propylene glycol and mannitol.

20. A production line for producing a freeze dried food product functionally impregnated with a material of interest, said production line comprising:
a. an instruction protocol for operating said production line
b. a lyophilizer adapted for freeze-drying a food product;
c. an incubator adapted for incubating said food product in solution containing material of interest under vacuum and further adapted for incubating with solution containing taste additives under normal pressure;
d. a drier adapted for drying the food product after an incubation step;
wherein said instruction protocol is encoded in a computer readable medium or written on printed matter or represented on a computer screen and said production line provides said food product substantially reduced in moisture to between about 0% and about 5%; said food product remaining in it's original structure and shape; further wherein said food product is functionally impregnated with said material of interest after applying said incubator.

21. A computer readable medium comprising instructions for causing a computer to operate a production line producing a freeze dried food product functionally impregnated with a material of interest, by the method comprising steps of:
a. freeze drying a fresh food product to remove moisture content;
b. incubating dried food product in a solution containing material of interest, and;
c. incubating of the wet food product with a soaked solution containing taste additives under normal pressure
wherein after step (b), said food product is substantially reduced in moisture to between about 0% and about 5%; said food product remains in it's original structure and shape; further wherein said food product is functionally impregnated with said material of interest after step d.
